(19) United States
(12) Patent Application Publication
(10) Pub. No.: US 2024/0024566 A1
(43) Pub. Date: Jan. 25, 2024

(54) Delivery Device Systems, Methods, and Apparatuses

(71) Applicant: DEKA Products Limited Partnership, Manchester, NH (US)

(72) Inventors: Dean Kamen, Bedford, NH (US); Lee A. Batchelder, Auburn, NH (US)

(21) Appl. No.: 18/197,962

(22) Filed: May 16, 2023

Related U.S. Application Data
(60) Provisional application No. 63/390,992, filed on Jul. 21, 2022.

(51) Int. Cl. A61M 5/142; A61M 5/14

(52) U.S. Cl. CPC A61M 5/142; A61M 5/1408; A61M 39/22

(57) ABSTRACT

An access assembly for administration of agent to a shallow delivery destination may comprise a body having a first exterior surface and a second exterior surface. The first exterior surface may be at a non-orthogonal angle to the second exterior surface. The body may have a passage extending therethrough to a corner formed between the first and second exterior surfaces. The assembly may further comprise an adhesive pad coupled to the second exterior surface. The assembly may further comprise a member having a flow path extending therethrough and a sharp bearing body including a number of microneedles coupled to an end of the member. The member may be disposed within the passage and in contact with at least one stop defined by the passage. The assembly may further comprise a coupler configured to couple with a cooperating coupler on a fluid flow conduit.

### CROSS-REFERENCE TO RELATED APPLICATIONS

The present application claims the benefit of U.S. Provisional Application Ser. No. 63/390,992 filed Jul. 21, 2022, and entitled Delivery Device Systems, Methods, and Apparatuses (Attorney Docket No. 00101.00334.AA923), which is hereby incorporated herein by reference in its entirety.

### STATEMENT REGARDING FEDERALLY SPONSORED RESEARCH OR DEVELOPMENT

This invention was made with Government support under Agreement W911NF-17-3-0003, awarded by ACC-APG-RTP. The Government has certain rights in the invention.

### BACKGROUND

### Field of Disclosure

This disclosure relates to medical agent delivery. More specifically, this disclosure relates to dispensers for medical agents.

### Description of Related Art

Shallow delivery (e.g. intradermal administration) of agents to patients is typically performed via Mantoux technique. In this technique, a standard small gauge needle is manually inserted into skin at a shallow angle and agent is delivered intradermally via a syringe. This technique, however, is reliant upon a caregiver to appropriately position the outlet of the needle at a suitable depth and deliveries via Mantoux technique can be challenging to perform correctly even for trained professionals. Leaks from the injection site or delivery to destinations deeper than the target destination can occur. Despite this, shallow agent delivery is recognized to have a variety of potential benefits and may open up new avenues for treatment of various conditions. Thus, a shallow agent delivery platform which can reliably be used to administer to a shallow delivery destination without need for a skilled professional is desired.

### SUMMARY

In accordance with an embodiment of the present disclosure an access assembly for administration of agent to a shallow delivery destination may comprise a body having a first exterior surface and a second exterior surface. The first exterior surface may be positioned at a non-orthogonal angle to the second exterior surface. The body may have a passage extending therethrough and to a corner formed between the first and second exterior surfaces. The passage may define at least one stop. The access assembly may further comprise an adhesive pad coupled to the second exterior surface. The access assembly may further comprise a member having a flow path extending through the member. A sharp bearing body from which a number of microneedles project may be coupled to an end of the member. The member may be disposed within the passage and in contact with the at least one stop. The at least one stop may be configured to inhibit displacement of the member within the passage beyond a position in which sharp bearing body is even with the first exterior surface. The access assembly may further comprise a coupler configured to couple with a cooperating coupler on a fluid flow conduit.

In some embodiments, the angle between the first and second exterior surfaces may be 10-50°. In some embodiments, the passage has may have a “t” shaped or cruciform cross-section. In some embodiments, the coupler may be defined on an end of the member opposite the end to which the sharp bearing body is coupled. In some embodiments, the member may be interference fit into the passage. In some embodiments, the coupler may be a luer fitting. In some embodiments, the microneedles may have a height between 200-1500 microns. In some embodiments, the number of microneedles may be a one dimensional array of microneedles. In some embodiments, the cross-sectional area of the passage may change along the axis of the passage. In some embodiments, the sharp bearing body may form an extension of the first exterior surface when the member is in contact with the stop.

In accordance with another embodiment of the present disclosure a system for administration of agent to a shallow delivery destination may comprise an access assembly. The access assembly may comprise a body with a first exterior surface and a second exterior surface at a non-orthogonal angle to the first exterior surface. The first and second exterior surface may meet at a corner of the body. The access assembly may further comprise an adhesive pad coupled to the second exterior surface. The access assembly may further comprise a sharp bearing body having at least one microneedle projecting therefrom. A surface of the sharp bearing body from which the at least one microneedle projects may be substantially even with the first exterior surface. The system may further comprise an infusion device in fluid communication with the access assembly. The infusion device may comprise a reservoir. The infusion device may further comprise a pumping arrangement operable to deliver fluid form the reservoir to the access assembly. The infusion device may further comprise a controller configured to govern operation of pumping assembly to deliver at least one predefined volume of agent from the reservoir to the access assembly at at least one predefined rate over at least one predefined period of time.

In some embodiments, the body may include a passage extending through the sharp bearing body. The sharp bearing body may be coupled to a member disposed within the passage. The member may have a flow path extending to the sharp bearing body through the member. In some embodiments, the member may be interference fit within the passage. The passage may include a stop which inhibits displacement of the member within the passage beyond a certain point. In some embodiments, the infusion device may be in fluid communication with the access device via a run of tubing. In some embodiments, the at least one microneedle may include a one dimensional array of a plurality of microneedles. In some embodiments, the at least one microneedle has a height of less than 800 microns. In some embodiments, the at least one microneedle may have a height dimension of 200-1500 microns. In some embodiments, the infusion device may include a volume sensing assembly configured to collect data related to the volume of agent dispensed from the infusion device. In some embodiments, the access assembly may include a coupler configured to couple to a cooperating coupler in fluid communication with the infusion device. In some embodiments, the infusion device may include a reusable portion and a cassette coupled to the reusable portion. The reusable portion may include the controller and a first portion of the pumping arrangement. The cassette may include the reservoir and a second portion of the pumping arrangement. The second portion of the pumping arrangement may include all components of the pumping arrangement which contact agent as the agent is dispensed.

In accordance with another embodiment of the present disclosure a method of delivering an agent to a shallow delivery destination may comprise placing an infusion device in fluid communication with an access assembly including at least one microneedle. The method may further comprise displacing the access assembly against a barrier in a puncture position in which a first exterior surface of the access assembly is pressed against the barrier and the at least one microneedle extends into the barrier. The method may further comprise tilting the access assembly to a mounted position in which an adhesive pad coupled to a second exterior surface of the access assembly is in an adhering relationship with the barrier. The second exterior surface may meet the first exterior surface at a corner. The method may further comprise governing operation of a pumping arrangement of the infusion pump, via a controller, to deliver agent from a reservoir of the infusion device out of the at least one microneedle.

In some embodiments, placing the infusion device in fluid communication with the access assembly may comprise coupling a coupler associated with the infusion device to a cooperating coupler associated with the access assembly. In some embodiments, a height dimension of the at least one microneedle may be disposed substantially normal to the barrier when the access assembly is in the puncture position. In some embodiments, tilting the access assembly may comprise tilting the access assembly 10-50° degrees. In some embodiments, tilting the access assembly may comprise displacing the at least one microneedle in a non-straight path within the barrier. In some embodiments, the at least one microneedle may extend beyond the footprint of the second exterior surface of the access assembly when the access assembly is in the mounted position. In some embodiments, governing operation of the pumping arrangement may comprise generating commands with the controller, the commands operating the pumping arrangement to deliver a predefined volume of agent. In some embodiments, governing operation of the pumping arrangement may further comprise generating commands with the controller, the commands operating the pumping arrangement to deliver the predefined volume of agent at a predefined rate. In some embodiments, governing operation of the pumping arrangement may further comprise generating commands with the controller, the commands operating the pumping arrangement to deliver the predefined volume over a predefined period of time. In some embodiments, governing operation of the pumping arrangement may further comprise generating commands with the controller, the commands operating the pumping arrangement to deliver the agent based on a predefined schedule.

### BRIEF DESCRIPTION OF THE DRAWINGS

FIG. 1 depicts a block diagram of an exemplary system including an example access assembly and an example infusion device;

FIG. 2 depicts a perspective view of an example embodiment of an access member;

FIG. 3A-3B depict views of an example embodiment of an access member;

FIG. 4A depicts a view of an example embodiment of a number of access members on an exemplary sharp bearing body;

FIG. 4B depicts a detailed view of an indicated region of the indicated region of FIG. 4A;

FIG. 5A depicts a perspective view of an example embodiment of a number of access members on an example sharp bearing body;

FIG. 5B depicts a perspective view of an example embodiment of a number of access members on an example sharp bearing body;

FIG. 6A depicts a perspective view of an example embodiment of a number of access members on an example sharp bearing body;

FIG. 6B depicts a top plan view of an example embodiment of a number of access members on an example sharp bearing body;

FIG. 7A depicts a top plan view of an example embodiment of a number of access members on an example sharp bearing body;

FIG. 7B depicts a perspective view of an example embodiment of a number of access members on an example sharp bearing body;

FIG. 8A depicts a top plan view of an example embodiment of a number of access members on an example sharp bearing body;

FIG. 8B depicts a perspective view of an example embodiment of a number of access members on an example sharp bearing body;

FIG. 8C depicts a cross-sectional view taken at the indicated cut plane of FIG. 8A;

FIGS. 9A-9D depict various views of an example access member;

FIGS. 10-15 depict block diagram views of example delivery assemblies which may be included in certain example infusion devices;

FIG. 16 depicts an example access assembly;

FIG. 17A depicts a cross-sectional view of an example access assembly in a puncture position on a barrier;

FIG. 17B depicts a cross-sectional view of an example access assembly in a mounted position on a barrier;

FIG. 17C depicts a detailed view of the indicated region of FIG. 17B; and

FIG. 18 depicts a flowchart detailing a number of example actions which may be executed to deliver agent via an example system including an access assembly and an infusion device.

These and other aspects will become more apparent from the following detailed description of the various embodiments of the present disclosure with reference to the drawings wherein:

### DETAILED DESCRIPTION

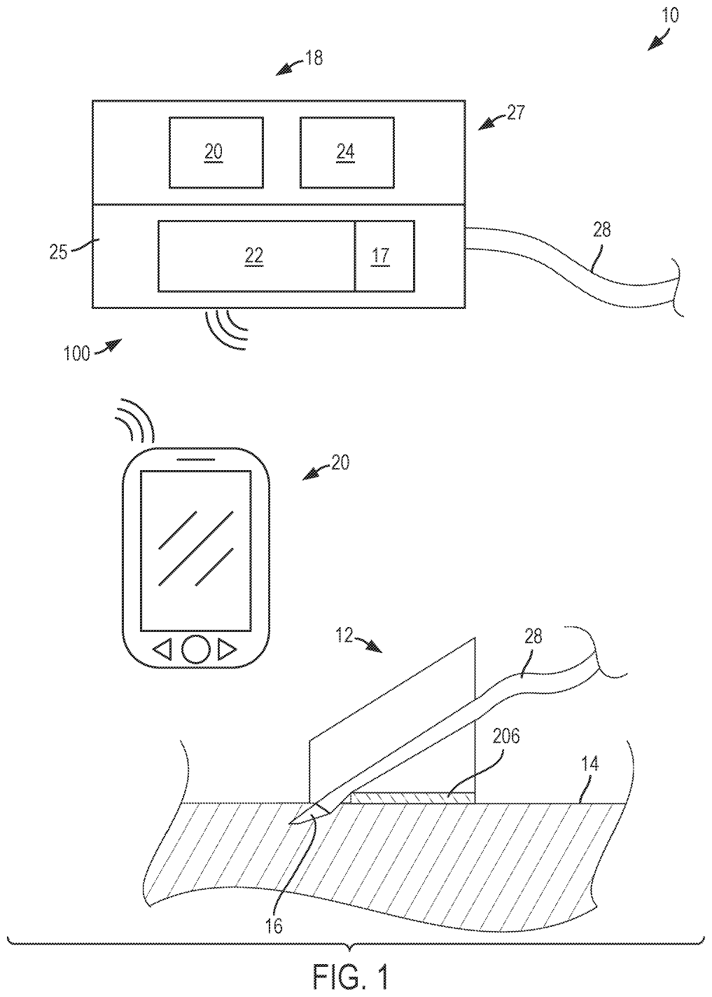

Referring now to FIG. 1, a block diagram of an exemplary system 10 is depicted. The exemplary system 10 may include an access assembly 12. The access assembly 12 may be any of the access assemblies 12 described herein. The access assembly 12 may adhere to a barrier 14 which is present between the surrounding environment and a desired delivery destination. The access assembly 12 may establish access to a delivery destination in the patient. The access assembly 12 may include at least one access member 16 which may allow for fluid flow from the access assembly 12 out of each access member 16 and into the delivery destination. Each of the access members 16 may be an indwelling body which extends at least partially into the barrier 14 during use of the access assembly 12. In various examples, the barrier 14 may be an exterior surface of the skin of a subject. Where reference is made to skin herein it should be understood an access assembly 12 may be used in relation to other barriers 14 and reference to skin is merely exemplary. The subject may be a human, though in alternative embodiments, may also be other multicellular organisms. For non-limiting example, the subject may be a mammal, rodent, mouse, dog, primate, pig, etc. The delivery destinations may be a shallow delivery destination in certain examples. For instance, where the barrier 14 is an exterior surface of the skin 14, the at least one access member 16 may deliver fluid into a portion of the skin between the stratum corneum and subcutaneous tissue. Shallow delivery destinations may include an epidermal or dermal target location or may, for example, target a junctional area between the epidermis and dermis or dermis and subcutis. The delivery destination may be an intradermal delivery destination. The at least one access member 16 may, for example, be any of the access members 16 described herein and may include at least one delivery sharp such as a microneedle in certain examples.

Application of an access assembly 12 with access member(s) 16 for delivery into a shallow delivery destination may be painless as the access member(s) 16 may be too short to reach nerve endings which are located deeper in the anatomy. Additionally, certain types of access member(s) 16 may be better tolerated by patients. Silicon microneedles, for instance, may not have the same allergy concerns as access member(s) 16 formed from materials including nickel (e.g. stainless steel). Access assemblies 12 may further allow such delivery to be performed hands-free once the access assembly 12 has been applied to the barrier 14. Access assemblies 12 may also help allow for repeatable and reliable placement of the access member(s) 16 into communication with the delivery destination as access assemblies 12 may intuitively guide user placement of the access assembly 12 on the subject. Additionally, access assemblies 12 may inhibit displacement of the access member(s) 16 once the access member(s) 16 have been brought to a desired delivery position after puncture of the barrier 14.

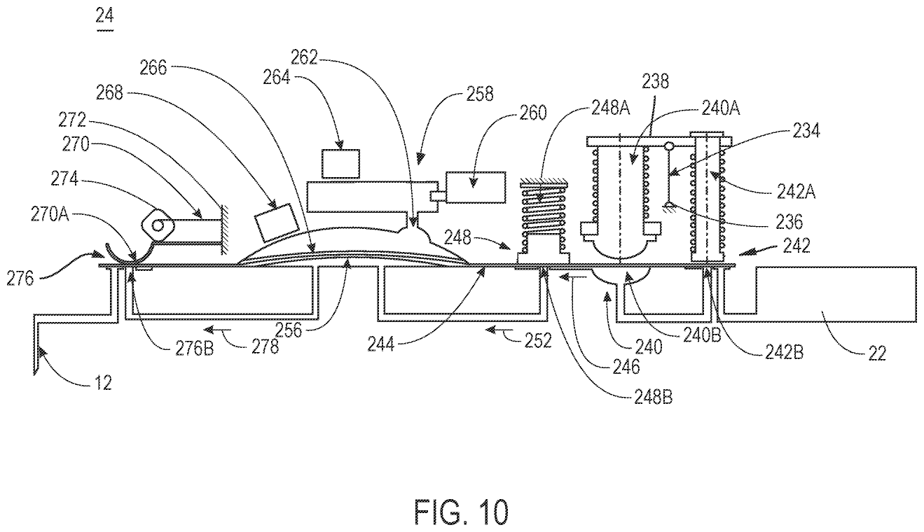

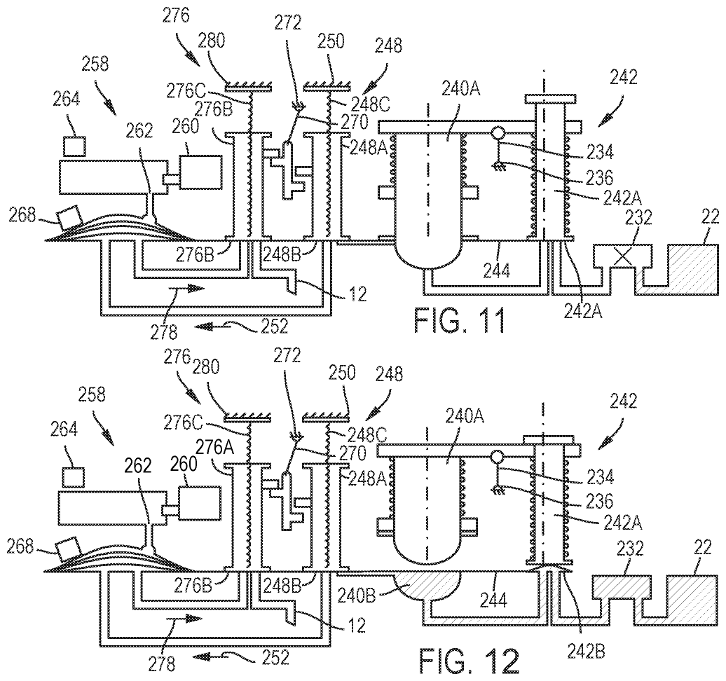

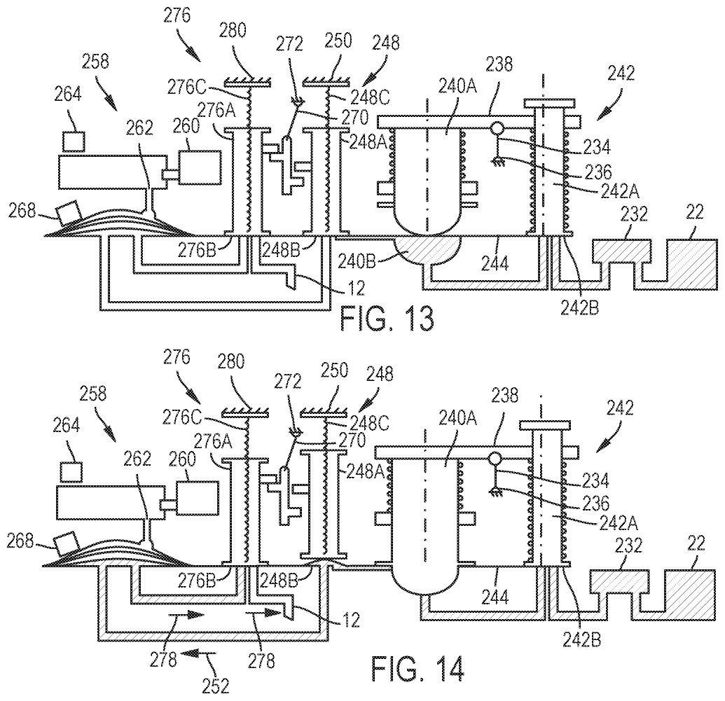

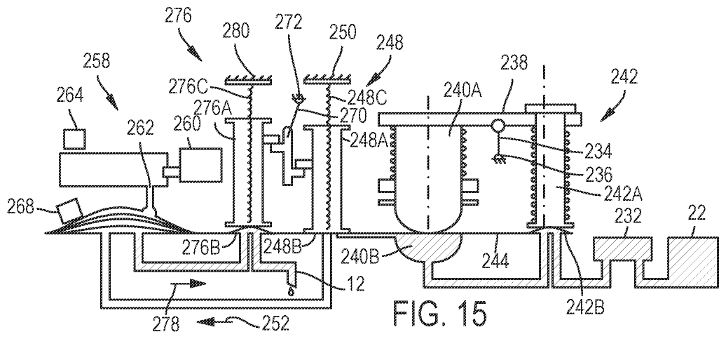

The access assembly 12 may fluidically communicate with an infusion device 18. The infusion device 18 may include a controller 20 which may govern operation of a delivery assembly 24 (e.g. pumping components, valves, sensors monitoring pumping components or configured to provide data related to aspects of fluid delivery from the infusion device, etc.) to output desired volumes of fluid from a reservoir 22 associated with the infusion device 18. Multiple controllers 20 may be included in certain embodiments and at least one of the controllers 20 may be disposed outside of the infusion device 18 and be in data communication (wired or wireless) therewith. For example, a controller 20 may be included in a smartphone, tablet, PC, laptop, or the like. An example delivery assembly 24 is depicted and described in relation to FIGS. 10-16.

In certain embodiments, the infusion device 18 may include a reusable component and a disposable component which may be removably coupled to one another. In the example shown in FIG. 1, the infusion device 18 includes a cassette assembly 25 which may attach to a reusable portion 27 of the infusion device 18. The cassette assembly 25 may include a reservoir 22 and may be replaced when fluid in the reservoir 22 has been depleted. The delivery arrangement 24 may be split between the cassette assembly 25 and the reusable portion. The cassette assembly 25 may, for example, include fluid pathways and valves which may be acted on through a flexible membrane overlaying at least a portion of the cassette 25. A septum 17 may also be included and may provide an access to the interior of the reservoir 22. The reusable portion 27 may include any of a controller 20, a power source (e.g. battery), a speaker, a user interface, wireless communication hardware, and various sensors and actuators to govern dispensing of fluid through the cassette assembly 25.

The infusion device 18 (e.g. an outlet of cassette assembly 25) may fluidically couple to the access assembly 12 via a connector (e.g. luer lock arrangement). In alternative embodiments, an outlet of a cassette assembly 25 may be hard plumbed to the access assembly 12 as shown. In the example embodiment, the infusion device 18 is in fluid communication with the access assembly 12 via a run of tubing 28, however, in alternative embodiments, infusion device 18 may be connected directly to the access assembly 12. Where a luer arrangement is used, a luer lock fitting of the access assembly 12 (or in fluid communication therewith via a run of tubing 28) may engage with a luer fitting in fluid communication with the infusion device 18 (e.g. at the terminal end of a run of tubing leading from the cassette 25).

The infusion device 18 may deliver any desired fluid to the delivery destination via the access assembly 12. In various examples, the infusion device 18 may deliver at least one medical agent. Agents supplied may include drugs which are generally supplied as a continuous or substantially continuous infusion though other drugs may also be used. This may include small molecules, biologicals, recombinantly produced pharmaceuticals, and analogs thereof. In various examples, the infusion device 18 may deliver an agent which affects the cardiovascular system or blood vessels. For example, an infusion device 18 may deliver a vasodilator. In certain examples, a drug for the treatment of pulmonary arterial hypertension such as Treprostinil may be delivered. In some examples, an infusion device 18 may deliver a peptide such as a regulatory hormone. In some examples, the agent may be a drug for the treatment of diabetes or a drug which acts to alter blood glucose levels. In certain examples, the infusion device 18 may deliver insulin. In certain embodiments, an infusion device 18 may deliver glucagon. Chemotherapy drugs may also be delivered via the access assembly 12. In certain embodiments, agents may include agents used for medical or biological research. Where references to a particular agent are made in relation to examples described herein, their use is merely exemplary and it shall be understood, that use for other medical conditions or with other agents is contemplated.

Infusion devices include any infusion pump and may include, but are not limited to, the various infusion devices and components thereof described in U.S. patent application Ser. No. 13/788,260, filed Mar. 7, 2013 and entitled Infusion Pump Assembly, now U.S. Publication No. US-2014-0107579, published Apr. 17, 2014 (Attorney Docket No. K40); U.S. Pat. No. 8,491,570, issued July 23, 2013 and entitled Infusion Pump Assembly (Attorney Docket No. G75); U.S. Pat. No. 8,414,522, issued Apr. 9, 2013 and entitled Fluid Delivery Systems and Methods (Attorney Docket No. E70); U.S. Pat. No. 8,262,616, issued Sep. 11, 2012 and entitled Infusion Pump Assembly (Attorney Docket No. F51); and U.S. Pat. No. 7,306,578, issued Dec. 11, 2007 and entitled Loading Mechanism for Infusion Pump (Attorney Docket No. C54); all of which are hereby incorporated herein by reference in their entireties.

Microneedles described herein may include, but are not limited to, the various microneedles described in U.S. Pat. No. 11,154,698, issued Oct. 26, 2021, and entitled Microneedle Systems and Apparatus (Attorney Docket No. G34) or U.S. Pat. No. 5,983,136, issued Nov. 9, 1999, and entitled System for Delivery of Drugs by Transport (Attorney Docket No. B60).

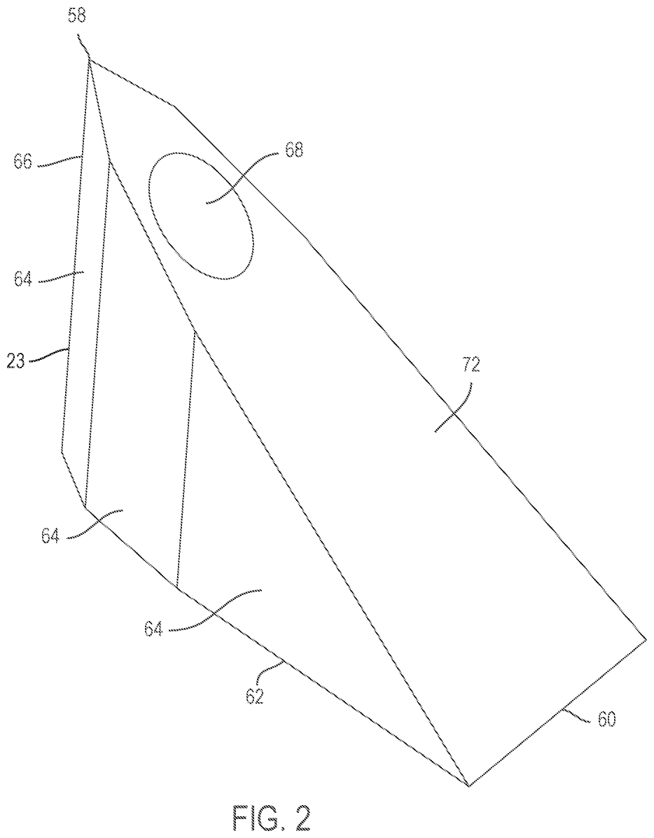

Referring now also to FIG. 2, where microneedles are used, the microneedles described herein may, in certain embodiments, be MEMS produced, polyhedral (e.g. pyramidal), silicon crystal microneedles. These microneedles may be no greater than 1 mm in height, e.g. 0.6 mm or 0.8 mm (though longer or shorter microneedles may also be used). In some embodiments, microneedles may be 1200-1500 microns in height or perhaps longer in some examples. The height of the microneedles used may be selected based on the intended subject and access assemblies 12 for different organisms may include microneedles with heights selected based on the depth of a desired delivery destination in that particular organism. In some embodiments, microneedles may have a height sufficient to puncture at least some distance into subcutaneous tissue. At least some edges of the microneedles may be rounded or filleted, though such microneedles may still be referred to herein as polyhedral. In some examples and as shown in FIG. 2, the microneedles described herein may be generally in the shape of a heptagonal prism (though pentagonal, nonagonal, and other polygonal prisms may also be used as the base shape) which has been diagonally sected to form a heptagonal ramp or pointed wedge. In such embodiments, the heptagonal prism may be sected by a plane extending from a vertex 58 of the top face of the prism through the most distal side 60 of the base 62. At least two sides of the base of the microneedle may be parallel. The side walls 64 may extend substantially perpendicularly from the base 62. A microneedle may be substantially symmetric about a line of symmetry extending from the vertex 58 to a point above the center of the most distal side 60. In other embodiments, a microneedle may be conically shaped. Any other suitable shape may be used. In the example, the vertex 58 is shown as a point which forms a tip of the microneedle. In other embodiments, this portion of a microneedle may be rounded (though may still be referred to herein as a vertex 58 and such microneedles may still be referred to as pointed). In such embodiments, the back facing edge 66 may be a round face or the back facing edge 66 and the adjacent side walls 64 may be replaced by a rounded face.

The points or tips of microneedles described herein may be solid and the flow lumens 68 through the microneedles may be offset from the points or tips (in FIG. 2 the vertex 58 forms the tip) of the microneedles. Hollow tipped microneedles in which the flow lumen 68 extends to the tip of the microneedle may also be utilized. In some embodiments, the microneedles may be NanoPass hollow microneedles available from NanoPass Technologies Ltd. of 3 Golda Meir, Nes Ziona, Israel. It should be noted that microneedles (or the substrate on which they are disposed) described herein as constructed of silicon may have a surface layer of silicon dioxide (which may, for example, form with exposure to air) while still being considered constructed of silicon.

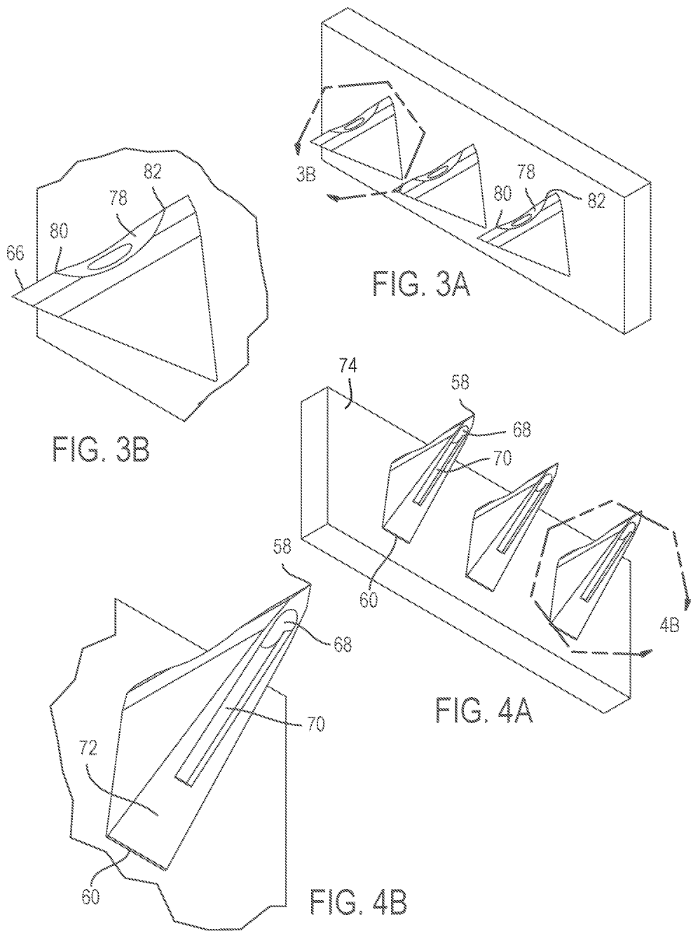

With reference to FIGS. 3A-4B, in some embodiments, microneedles may be constructed to include certain features that may help to reduce the pressure required to inject fluid, such as a medical agent, into the skin of a patient. In some examples, features common certain to insect stingers or biological venom administration structures may be incorporated. These features may include various recesses or depressions which are formed as part of each microneedle or at least one microneedle of an access assembly 12. These recesses or depressions may fluidly communicate with the flow lumen 68 of the respective microneedle. In some embodiments, different microneedles of an access assembly 12 may include different recesses or some microneedles may include a plurality of recesses which could be of different varieties (though need not be).

For example, as shown in FIGS. 3A-4B, a microneedle may include a channel or trough 70 on an exterior sloped face 72 leading from the flow lumen 68 toward the distal side 60. The channel 70 may allow medical agent to flow through it along the outer side of the microneedle to find a path of least resistance, or weakest link, into the skin. In the embodiments shown, medical agent may be routed by the channel 70 to flow along the outer side of the microneedle to a weak region in the skin in the event the outlet of the flow lumen 68 has been inserted to a greater depth than the depth of the weak region. The lamina lucida junction, an intradermal delivery destination, is a weak link in the skin structure, and is difficult to consistently inject directly into due to its relative thinness (it is typically on the order of 40 nm thick). A microneedle including a channel 70 may, for example, allow flow of medical agent to the lamina lucida junction when the lamina lucida junction has been passed by the outlet of the flow lumen 68. The channel 70 may facilitate distribution of the medical agent through a larger area of entry or injection and may help allow for delivery to occur at lower pressures.

An appropriate silicon etching technique (or mold in embodiments using polymeric microneedles) may be used to create steeper side walls of the channel 70. This may help inhibit the skin from bending into and occluding the channel 70. Etching techniques that could be used include, by way of non-limiting example, chemical etching techniques (e.g., acid). Suitable etching techniques may include ion based etching techniques (e.g. reactive ion etching). The etching process could be a wet etching process or a dry etching process. In some non-limiting embodiments, the channel 70 may be within a range of 50-60 microns wide from side to side. In some non-limiting embodiments, the flow lumen 68 may have a diameter of 50-60 microns. The channel 70 may have a width equal to the diameter or widest portion of the flow lumen 68 or the channel 70 may have a width which is less than or greater than the width of the flow lumen 68. In certain examples, the width of the channel 70 may be about 5-10 percent of the height of the microneedle.

To avoid leakage of the fluid from the channel 70, it may be desirable to ensure that the channel 70 terminates at least a certain distance beneath the surface of the skin yet also reaches the targeted skin layer (e.g., the lamina lucida junction) when the microneedle is inserted into the skin. In some embodiments the channel 70 extends from the flow lumen 68 to within at most 50 microns (e.g. 50-200 microns) of the base 62 of the microneedle. In some embodiments, the end of the channel 70 most proximal the base 62 of the microneedle may be at least below the stratum corneum (and perhaps one or more of the stratum lucidum, stratum granulosum, stratum spinosum, and stratum basale) when the microneedle is inserted into the skin. In some embodiments, the end of the channel 70 most proximal the base 62 may be disposed below the epidermis (e.g. in the basement membrane) or within the epidermis.

The channel 70 need not be straight or shaped in the manner shown in and described with reference to FIGS. 4A-4B. In some embodiments, the channel 70 may be a more meandering channel 70. A curved channel 70 could, for example, be used provided the dimensions of the microneedle are accommodated. Moreover, there need not be only one channel More than one channel 70 could be used provided structural integrity of the microneedle is accommodated.

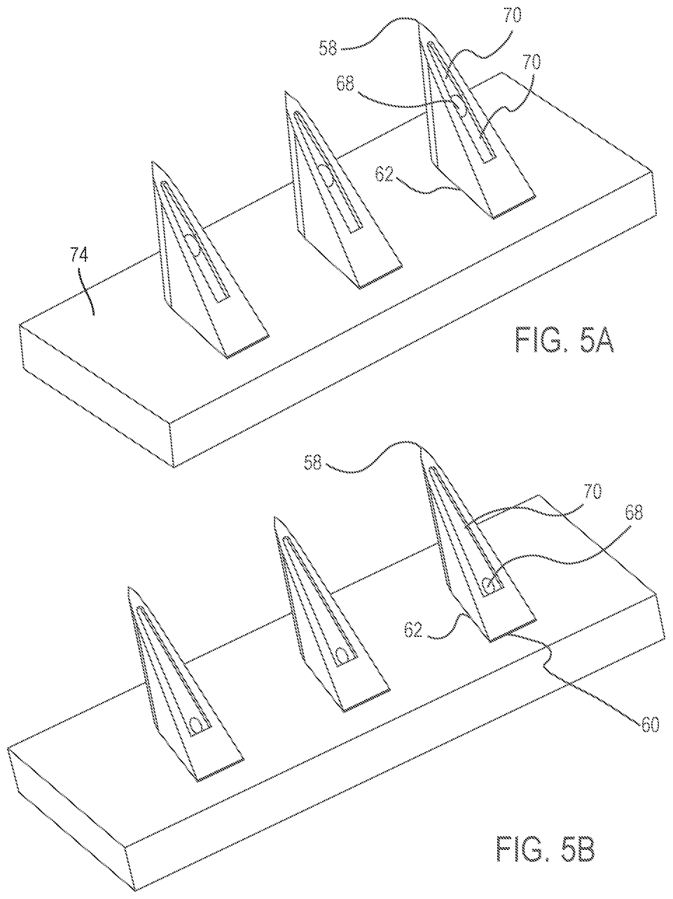

The depth of the channel 70 may be about 25 microns or more (e.g. 25-50 microns) in certain examples. The depth of the channel 70 may be or be less than 5 percent the height of the microneedle. While the depth of the channel 70 may be constant along the length of the channel 70, the depth of the channel 70 need not be constant along the length of the channel Likewise, the width of the channel 70 need not be constant along the length of the channel 70 (see, e.g., FIG. 5B). The width of the channel 70 may be about 20-30 percent of the width of the distal side 60 of the microneedle at the narrowest point in the channel 70. In some embodiments, the width of the channel 70 may increase as distance to the distal side 60 decreases. In some embodiments, at its widest, the channel 70 may have a width which is 50% or more the width of the distal side 60.

Referring now also to FIG. 5A and FIG. 5B, in other examples, the channel 70 may extend from the location of the lumen 68 toward the tip or vertex 58 of the microneedle (see, e.g., FIG. 5B). Moreover, in some examples, the channel 70 may extend both toward the vertex 58 and toward the base 62 from the location of the lumen 68. That is, the channel 70 may include a portion on both sides of the lumen 68 (see, e.g., FIG. 5A). As shown, the lumen 68 may be located substantially centrally in the sloped face 72 of the microneedle. In such embodiments, a channel 70 may extend toward the distal side 60 of the base 62 and a channel 70 may extend toward the tip or vertex 58. In other embodiments, the lumen 68 may be positioned at (or near) an end of the channel 70 most proximal the base 62.

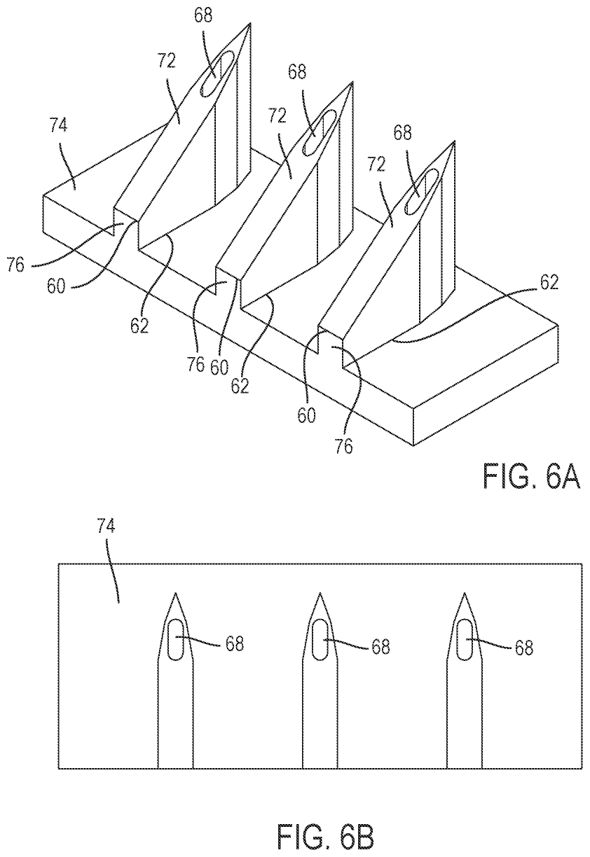

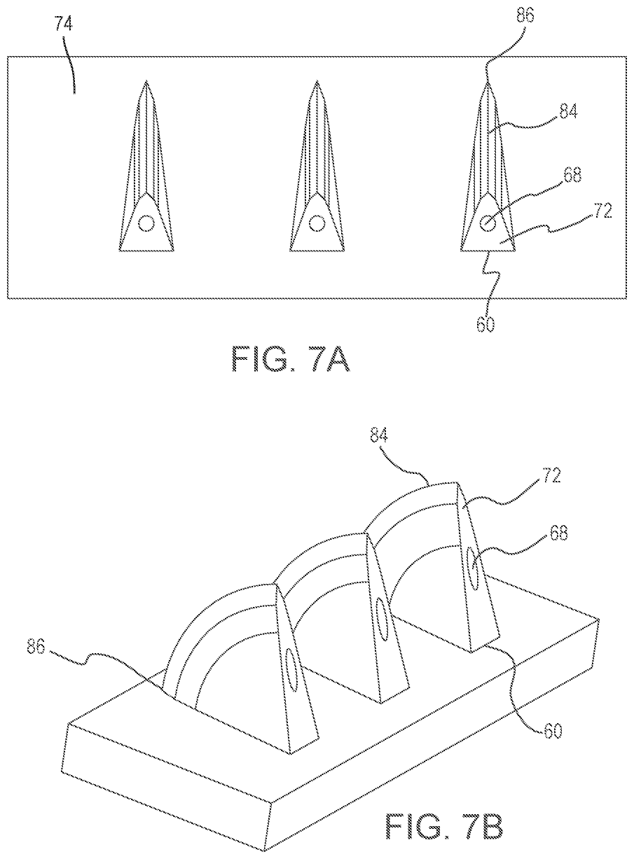

Referring now to FIGS. 6A-6B, views of a sharp bearing body 74 including a number of microneedles are shown. In certain embodiments, a channel 70 may not be included. Instead, a microneedle may include a flow lumen 68 with an elongate cross-section (at least at the outlet, see also FIGS. 7B & 8B). Microneedles with channels 70 and elongate lumens 68 are also possible. When in place within the patient, an elongate lumen 68 may be in fluid communication with, for example, multiple layers of skin. Thus, a thin and/or weak layer of skin may be easier to target when the microneedle is advanced into a patient. Elongate lumens 68 may also help to lower pressure required to inject. Such elongate flow lumens 68 may have any suitable cross-section. In some embodiments, the cross-section may be oval or elliptical. Alternatively, a lumen 68 with an obround cross-section may be used as is shown in FIGS. 6A-6B. Polygonal cross-sectional shapes may also be used, such as though not limited to rectangular, trapezoidal, triangular, etc. In certain examples, the length (in the direction of elongation) of the cross-section of the lumen 68 may be up to 100-200 microns or greater (though could be less in certain examples). Where elongate lumens 68 are included, the end of the lumen 68 most proximal the distal side 60 may be spaced from the distal side 60 by at least a certain distance. The spacing may be such that, the end of the lumen 68 most proximal the distal side 60 may be at least below the stratum corneum (and perhaps one or more of the stratum lucidum, stratum granulosum, stratum spinosum, and stratum basale) when the microneedle is inserted into the skin. In some embodiments, it may be disposed below the epidermis (e.g. in the basement membrane) or within the epidermis.

Still referring to FIGS. 6A-6B in certain embodiments, the sloped face 72 of a microneedle may not extend to the base 62 of a microneedle. There may, for example, be a vertical face 76 extending from the base 62 to the distal side 60 of a microneedle. Where a vertical face 76 is included, the vertical face 76 may be aligned with a side (e.g. distal side 60) of a sharp bearing body 74 and may form an extension thereof. Including such vertical faces 76 may aid in reducing the size of a sharp bearing body 74 and may aid in ensuring consistent fluid delivery into a target destination for certain microneedles. Though shown in relation to FIGS. 6A-6B, any of the microneedles shown herein may be arranged with vertical faces 76.

Additionally or in the alternative, a microneedle may include a depression 78. The depression 78 may include first and second opposing vertices 80, 82. In some embodiments the depression 78 may be (though need not necessarily be) a rounded depression or a concave depression, as shown in FIGS. 3A-3B. The depression 78 may have a maximum depth which places the depression 78 into fluid communication with the flow lumen 68 of the microneedle. The depression 78 may thus form a side port for the microneedle through which fluid may be delivered to the patient. The side port may be the only outlet of the microneedle or may be in addition to an outlet of the lumen in the sloped face 72 of the microneedle. When the microneedle is inserted into the skin surface, fluid transferred through an access assembly 12 may be delivered to the patient, at least in part, by being pumped into the depression 78. The depression 78 may be formed, for example by cutting away material during manufacture of the microneedle or the depression 78 may be formed during a molding operation. Cutting away material may be accomplished by any known suitable process such as, for instance, etching (e.g. wet etching). In some embodiments, the depression 78 may be recessed in at least one side wall 64 or edge (e.g. where two side walls 64 join) of the microneedle. In the example shown in FIGS. 3A-3B, the depression 78 is formed in a substantially vertical back facing edge 66 of the microneedles which extends from the base 62 to the vertex 58. This may establish or increase a vertical void volume created by the microneedle as the skin is penetrated by the microneedle. That is, such a depression 78 may establish an open space in a patient into which fluid may be easily delivered from the microneedle. Positioning the depression 78 in the back facing edge 66 may provide a path of low resistance for a fluid to enter skin that the microneedle has penetrated. In embodiments wherein the microneedle includes at least one substantially vertical wall, the depression 78 may be recessed into a substantially vertical wall. In the example embodiment, the maximum depth of the depression 78 may be about 130% to 110% of the distance from the back facing edge 66 to the flow lumen 68.

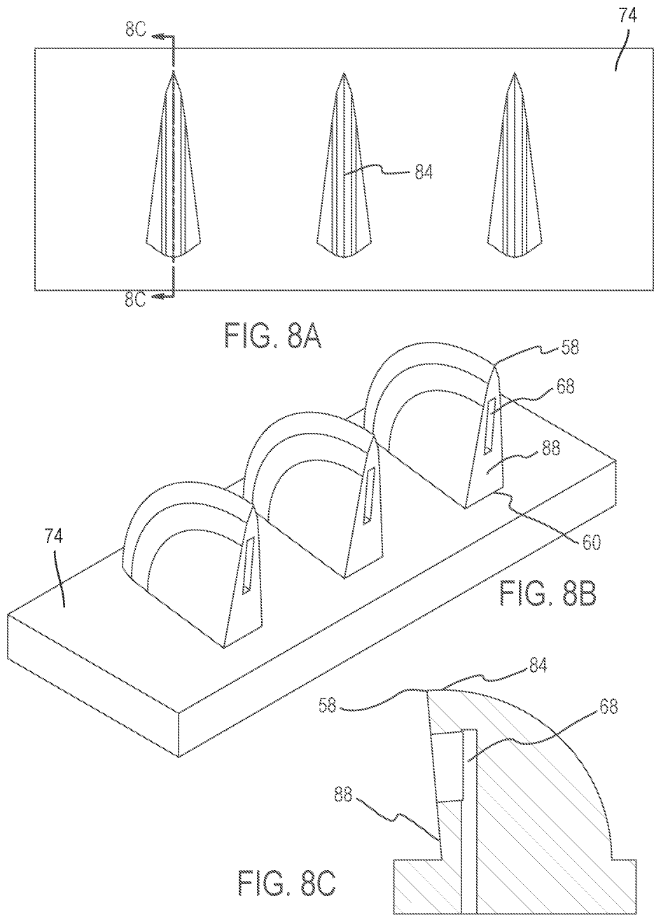

In certain examples, and referring now to FIG. 7A and FIG. 7B, a microneedle may include a sloped face 72 to which a lumen 68 extending through the microneedle extends. A microneedle may also include a rounded blade edge 84. In the example, the rounded blade edge 84 extends from a point 86 opposite the distal side 60 and extends in an arcuate path to the vertex or tip 58 of the microneedle. In the example, the rounded blade edge 84 includes a double bevel, though other bevel types may be used. The rounded blade edge 84 may arc at a constant radius or a variable radius. The rounded blade edge 84 may have an arc measure of less than 90° or, in certain examples, greater than 90° (see, e.g., FIGS. 8A-8C). The rounded blade edge 84 may aid in introduction of a microneedle into skin when the microneedle is inserted at certain angles or over a variety of different angles.

In yet another embodiment, and referring now to FIGS. 8A-8C, a microneedle may include a rounded blade edge 84 and a lumen outlet face 88. The lumen 68 may extend through the microneedle to the lumen outlet face 88 and may not be formed in a straight line through the microneedle. The lumen outlet face 88 may be angled from the vertex 58 to the distal side 60 so as to form an undercut. The distal edge 60 may be disposed such that a plane perpendicular to the base 62 passing through the distal edge 60 may also pass through the rounded or arcuate blade edge 84. Additionally, the outlet of the flow lumen 68 in the lumen outlet face 88 may be disposed such that a plane or all planes perpendicular to the base 62 and passing through the outlet of the flow lumen 68 may also pass through the blade edge 84. This need not be true in all embodiments (see, e.g., FIGS. 7A-7B). As a microneedle of the variety shown in FIGS. 8A-8B is inserted, a vertical void space may be created due to the undercut. This may provide a low resistance pathway for fluid injection. Additionally, the undercut may help to mitigate potential for the lumen 68 to become obstructed by skin as the microneedle is inserted into a patient or as the delivery occurs.

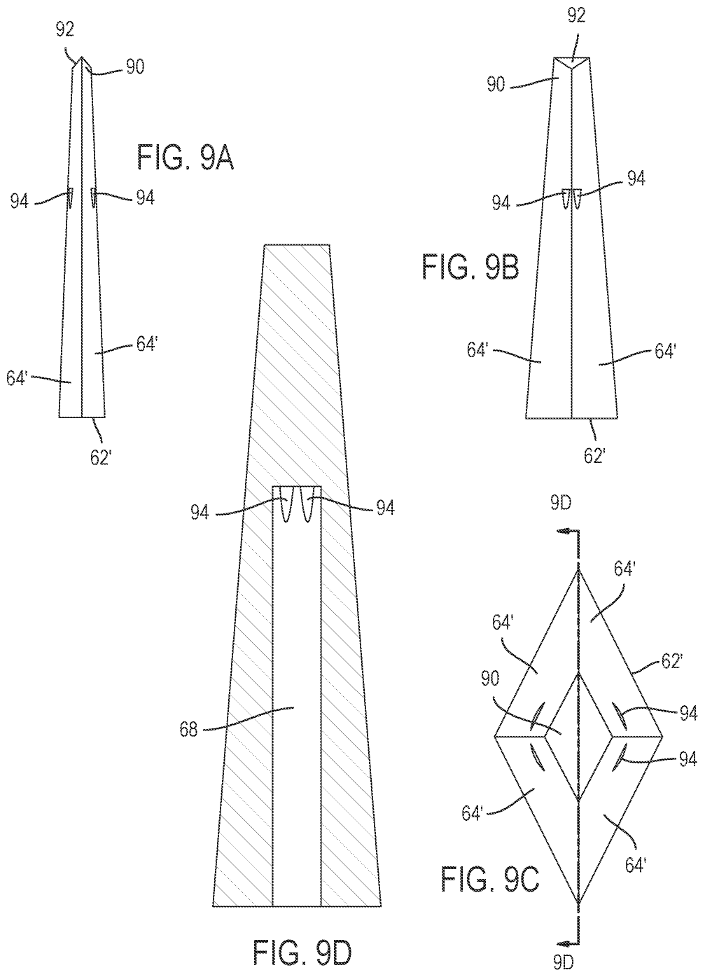

In still other embodiments and referring now to FIGS. 9A-9D, the access member(s) 16 may be or include a microneedle which has a shape with a high aspect ratio. In some embodiments, microneedles may be obelisk shaped. Such microneedles may be included in an array such as any array described herein. Where obelisk type microneedles are used, the microneedles may include a base 62′. The base 62′ may be any desired round or polygonal shape. For purposes of example, FIGS. 9A-9D depict a base 62′ which is a quadrilateral or rhombus. The example microneedle includes a set of sidewalls 64′ which extend from the base 62′ to an end region 90 of the microneedle. The sidewalls 64′ may be disposed at an angle which is not perpendicular to the base 62′. Thus the microneedle may taper so as to have a smaller cross-sectional area as distance from the base 62′ increases. A portion of the microneedle most distal to the base 62′ may include a beveled tip 92. Such a tip 92 may facilitate puncture of the skin and may aid in increasing the robustness of the end region 90. Any suitable bevel such as a single or double bevel may be used.

In embodiments of microneedles which are obelisk shaped, the microneedles may include at least one side port 94 which may serve as an outlet for that microneedle. Such side port(s) 94 may be difficult to block off with tissue which may become compressed during insertion of the microneedle into a patient. In the example embodiment, a lumen 68 may extend through the base 62′ of the microneedle and have a terminal end which is more proximal the end region 90 than the base 62′. The lumen 68 may be of relatively constant cross-section. The taper of the sidewalls 64′ may be such that the terminal end of the lumen 68 is wider than portions of the cross-section of the corresponding region of the microneedle. Thus, the lumen 68 may form openings in the sidewalls 64′ which may serve as the side ports 94. In various examples, the lumen 68 may be centrally disposed yielding symmetrical side ports 94. In alternative embodiments, the lumen 68 need not be centrally disposed and the side ports 94 may not be symmetrical.

Microneedles and features thereof may be manufactured in one or more of, though are not limited to, a molding process, etching process, ablative process (e.g. laser ablation), or a material additive process (e.g. 3D printed). In various embodiments, it may be desirable that microneedles be constructed of a biocompatible, non-ductile, high Young's modulus material with an indentation hardness sufficient to allow penetration into skin without breakage.

Access assemblies 12 including microneedles such as any of those described herein may be painless or nearly pain free when applied to a patient. This may make such access assemblies 12 user preferable over other types of delivery apparatuses, particularly with certain patient populations (e.g. juveniles). Additionally, access assemblies 12 described herein may be less complicated to apply and use (further described later in the specification).

Referring now to FIG. 10-15, a delivery assembly 24 or arrangement which may be included in a delivery device 18 (see, e.g., FIG. 1) is shown. The delivery assembly 24 depicted is an exemplary delivery assembly 24 and delivery devices 18 may include any of a variety of delivery arrangements 24. Certain delivery devices 18 may be syringe pumps. Certain delivery devices 18 may include peristatic pumping mechanisms (e.g. linear or finger type pumping mechanism or rotary peristaltic mechanism).

In the example delivery assembly 24, an occluder assembly 232 may isolate a filled reservoir 22 from the delivery assembly 24. Opening of the occluder assembly 232 may allow fluid to flow into the remainder of the delivery assembly 24. In order to effectuate the delivery of fluid within the reservoir 22 to the user, a controller 20 (see, e.g., FIG. 1) included within a delivery device 18 may command energizing of a shape memory actuator 234, which may be anchored on one end using a shape memory actuator anchor 236. An opposing end of the shape memory actuator 234 may be coupled to a common connector 238 attached to a pump plunger 240A and reservoir valve assembly 242. Energizing of the shape memory actuator 234 may result in the activation of a pump 240 and the reservoir valve assembly 242. The reservoir valve assembly 242 may include a reservoir valve actuator 242A and a reservoir valve 242B. Activation of the reservoir valve assembly 242 may result in the downward displacement of the reservoir valve actuator 242A and the closing of the reservoir valve 242B, resulting in the effective isolation of the reservoir 22 from the delivery assembly 24. A membrane 244 may be included between a pump plunger 240A and a pump chamber 240B of the pump 240. The reservoir valve actuator 242A may press the membrane 244 against a valve seat of the reservoir valve 242B in order to close the reservoir valve assembly 242. Pump 240 and reservoir valve assembly 242 may be arranged and connected by the connector 238 whereby reservoir valve assembly 242 may close prior to the pump 240 pumping fluid. The activation of the pump 240 may result in the pump plunger 240A being displaced in a downward fashion into the pump chamber 240B leading to a displacement of the fluid (in the direction of arrow 246). The pump chamber 240B may be shaped to be substantially the same as the end of the pump plunger 240A in order to substantially empty the pump chamber 240B with each stroke of the pump 240.

A volume sensor valve assembly 248 may include a volume sensor valve actuator 248A and a volume sensor valve 248B. Referring also to FIG. 12, the volume sensor valve actuator 248A may be maintained in a closed position via a volume valve spring assembly 248C (e.g. acting against a spring anchor 250) that provides mechanical force to move the volume sensor valve actuator 248A against the volume sensor valve 248B to seal volume sensor valve 248B. The volume sensor valve actuator 248A may press a membrane 244 included in the cassette assembly 25 against a valve seat of the volume sensor valve 248B in order to close the volume sensor valve 248. When the pump 240 is activated, however, if the displaced fluid is of sufficient pressure to overcome the mechanical sealing force of the volume sensor valve assembly 248, displacement of the fluid may occur in the direction of arrow 252. This may result in the filling of a volume sensor chamber 256 included within a volume sensor assembly 258 (shown in FIG. 14). Through the use of a speaker assembly 260, port assembly 262, reference microphone 264, spring diaphragm 266, and variable volume microphone 268, the volume sensor assembly 258 may determine the volume of fluid within the volume sensor chamber 256. Operation of such a volume sensor assembly 258 may be as discussed in, for example, U.S. Pat. No. 8,491,570 issued Jul. 23, 2013 and entitled Infusion Pump Assembly (Attorney Docket No. G75) which is incorporated herein by reference in its entirety above. Other suitable dispensed volume sensors may be used in other embodiments.

Referring also to FIG. 14, a shape memory actuator 270 may be anchored (on a first end) to a shape memory actuator anchor 272. Additionally, the other end of the shape memory actuator 270 may be used to provide mechanical energy to a valve actuator 274, which may activate a measurement valve assembly 276. Once the volume of fluid included within the volume sensor chamber 256 is calculated, the shape memory actuator 270 may be energized, resulting in the activation of measurement valve assembly 276. The measurement valve assembly 270 may include a measurement valve actuator 276A and a measurement valve 276B. Once activated to lift the measurement valve actuator 276A from the measurement valve 276B, due to the mechanical energy asserted on the fluid within volume sensor chamber 256 by the spring diaphragm 266, the fluid within the volume sensor chamber 256 may be displaced (in the direction of arrow 278) through access assembly 12 and into a subject. The measurement valve actuator 276A may then, by de-energizing the shape memory actuator and by action of the measurement valve spring assembly 276C (e.g. acting against spring anchor 280) press a membrane included in the cassette assembly 25 against a valve seat 276B in order to close the measurement valve 276B. In some embodiments, the membrane interfaces 244 included over the reservoir valve 242B, pump chamber 240B, volume sensor valve 648B, and the measurement valve 276B may be formed in a single piece of material having regions overlying each of these components.

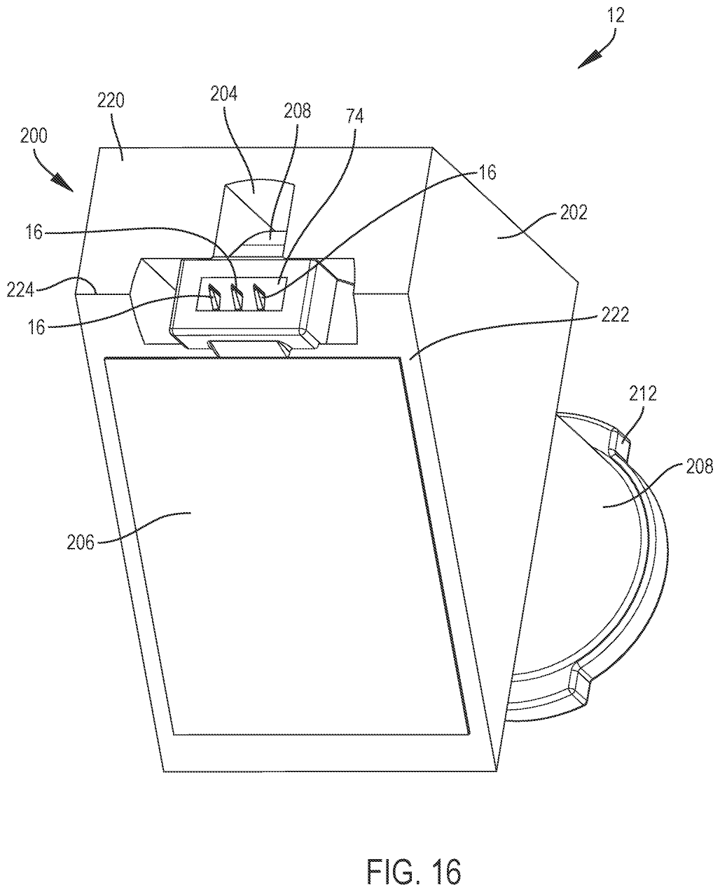

Referring now to FIG. 16, an exemplary embodiment of an access assembly 12 is depicted. As shown, the access assembly 12 includes an adapter 200. The adapter 200 is formed by a body 202 having a passage 204 extending therethrough. In the example embodiment, the body 202 is a monolithic piece of material (e.g. plastic) though in other embodiments may be formed by a plurality pieces which are coupled together. In the example embodiment, the body 202 has a quadrilateral cross sectional shape, in particular a trapezoidal cross-sectional shape. Other polygonal cross-sectional shapes may alternatively be used. In other examples, the body 202 may have a cross-sectional shape with at least one and preferably two flat sides. The passage 204 may have one end that extends to a corner 224 of the body 202 (or a transition region between the flat side and a rounded portion of the body 202). In certain examples, a rounded edge as opposed to an angled corner 224 may be included. In such embodiments, this feature may still be referred to as corner 224. The passage 204 may have any suitable cross sectional shape. In the example, the cross-section of the passage 204 is in the shape of the Latin character “t” or is cruciform in shape. As shown, the adapter 200 may further include an adhesive pad 206. The adhesive pad 206 may be coupled to a flat side of the body 202. The adhesive pad 206 may include a skin compatible adhesive. The adhesive pad 206 may be covered in a peelable backing (now shown) which may be removed to expose the adhesive on the adhesive pad 206 prior to use.

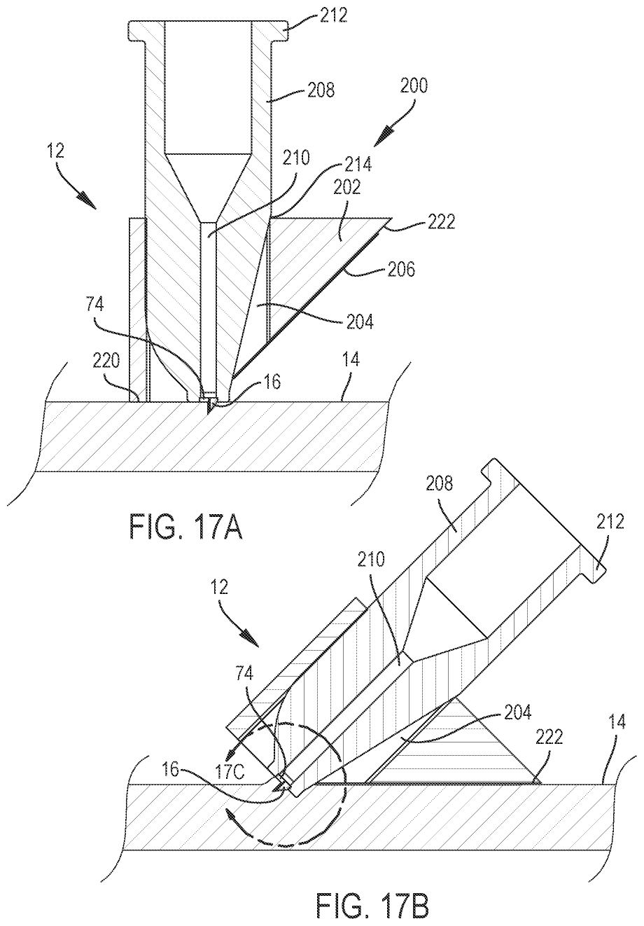

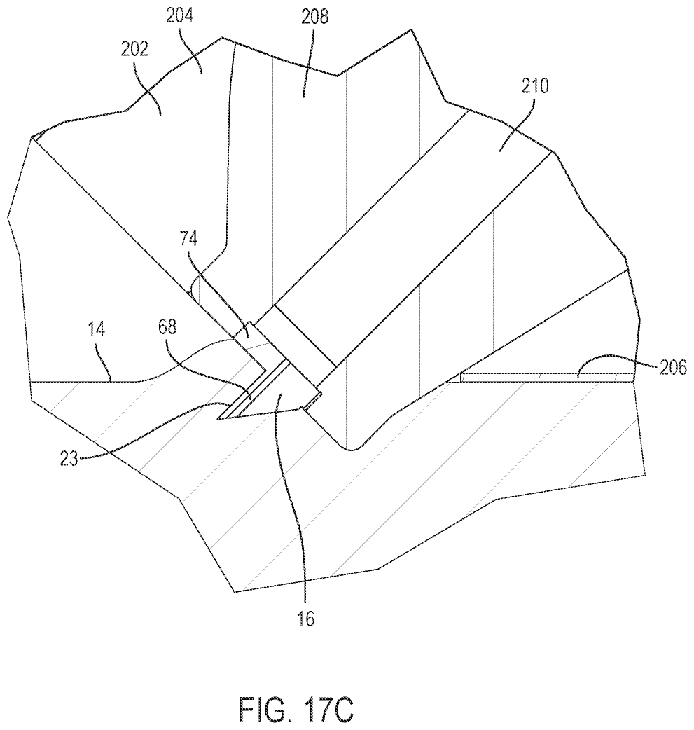

Still referring to FIG. 16, the access assembly 12 may include a member 208 bearing the access member(s) 16 of the access assembly 12. In the example embodiment, the access assembly 12 includes three access members 16 in a one dimensional array. The example access members 16 are depicted as microneedles similar to that shown in FIG. 2. The microneedles are defined as projections on a sharp bearing body 74. In alternative embodiments, there may be a greater or lesser number of microneedles and the microneedles may be arranged in any number of rows and columns. The sharp bearing body 74 may be coupled to an end of the member 208 and may be in fluid communication with a bore 210 (see, e.g., FIG. 17A) extending through the member 208. As shown, the member 208 may include a coupler 212, for example, at an end of the member opposite the access members 16. The coupler 212 may be a male or female portion of a luer arrangement in various examples. The coupler 212 may mate with a corresponding coupler on an infusion device 18 (see, e.g., FIG. 1) or a run of tubing 28 (see, e.g., FIG. 1) extending from an infusion device 18.

In certain alternative embodiments, the member 208 may be omitted and the access member(s) 16 may be mounted directly to the adapter 200 (e.g. via an overmolding process). For example, the body 202 of the adapter 200 may include a pocket or platform to which a sharp bearing body 74 with a number of microneedles may be coupled. In such examples, a fluid flow path may extend through the body 202 and into communication with lumens 68 (see, e.g., FIG. 4A) of the access member(s) 16. The coupler 212 may form or be coupled to a part of the body 202 in such examples. In some examples, the coupler 212 may be at a terminal end of a run of tubing coupled to the flow path extending through the body 202.

In some embodiments, a cap (not shown) may be included and may cover the microneedles. The cap may be removed prior to use. In some embodiments, the cap may be coupled to the adhesive backing covering the adhesive pad 206. Removal of the adhesive backing may also remove the cap for the microneedles.

The body 202 may have a width which is 3-4 times the width of the sharp bearing body 74. Where an array of microneedles is included, the width of the body 202 may be 5-7 times the longest distance between microneedles at the edges of the array.

Referring now to FIG. 17A, a cross-sectional view of the example access assembly 12 of FIG. 16 is depicted. The access assembly 12 is shown in a puncture position where the access member(s) 16 have penetrated the barrier 14. As shown, the example body 202 includes a first exterior surface 220 and a second exterior surface 222. The first exterior surface 220 and second exterior surface 222 may be disposed at a predefined angle to one another. The predefined angle may be an angle other than an orthogonal angle. The predefined angle may, for example be 10-80° in various embodiments. In the example embodiment, the angle is about 45° (the interior angle forming the corner 224 of the body being about 135°).

As shown, the passage 204 in the body 202 may define at least one stop surface 214. The stop surface(s) 214 may be positioned to inhibit displacement of the member 208 through the passage 204 once the member 208 has been advanced into the passage 204 by more than a certain distance. Thus, the at least one stop surface 214 may allow for repeatable positioning of the access member(s) 16 when the member 208 is installed into the passage 204. The passage 204 may be sized to create an interference fit once the member 208 is installed within the passage 204. The stop surfaces 214 may be positioned such that advancement of the member 208 into the passage is halted once the surface of a sharp bearing body 74 from which the access member(s) 16 extend is substantially even with the exterior surface of a side of the body 202. In the example embodiment, this sharp bearing body 74 surface is even with the first exterior surface 220 of the body 202. Thus, the first exterior surface 220 and the surface of the sharp bearing body 74 may bottom out on the barrier 14 and limit the puncture depth achieved by the access member(s) 16 when the access assembly 16 is in the puncture position. The passage 204 may be positioned such that when the member 208 is installed in the body 202 the access member(s) 16 project substantially from the plane of the first exterior surface 220, but from a point outside the periphery of the first exterior surface 220. When installed the end of the member 208 to which the access member(s) 16 are coupled may form an extension of the first exterior surface 220 which extends from the corner 224 where the first and second exterior surfaces 222 meet.

Referring now to FIGS. 17A and 17B, to place the access assembly 12 on a subject, the access assembly 12 may first be displaced against the barrier 14 in a puncture position (see FIG. 17A). In the puncture position, the height dimension of the access member(s) 16 may be substantially normal to the barrier 14. The access assembly 12 may be displaced against the barrier 14 such that the first exterior surface 220 abuts the barrier 14. As the access member(s) 16 extend proud of the first exterior surface 220, the access member(s) 16 may puncture the barrier 14. With the access member(s) 16 penetrating the barrier 14, the access assembly 16 may then be tilted until the second exterior surface 222 is parallel to the barrier 14 and the adhesive pad 206 is affixed to the surface of the barrier 14. The adhesive pad 206 may cover a majority of the second exterior face 222. The access assembly 16 may be considered to be in a mounted position when the adhesive pad 206 has been displaced into an adhering relationship with the barrier 14. The access assembly 12 may be maintained in the mounted position by the interaction of the adhesive pad 206 with the barrier 14. The width of the body 202 may be selected to help resist roll motion of the adapter 200 when the adapter 200 is in the mounted position. Additionally, when in the mounted position, no manual pressure may be needed to hold the access assembly 12 in place. Depending on the orientation of the barrier 14, the gravitation pull acting on the access assembly 12 may tend to pull the barrier 14 away from underlying structures. Thus compression of the delivery destination, which may tend to make delivery more challenging, may be substantially avoided.

Referring now also to FIG. 17C, tilting of the access assembly 12 may result in displacement of the access member(s) 16 in a non-straight path within the barrier 14. The access member(s) 16 may rotate or swing in an arcuate path as the access assembly 12 is brought to the mounted position. The access member(s) 16 may be considered to be in a delivery position when the access assembly 12 has been transitioned to the mounted position. In the delivery position the access member(s) 16 may extend beyond the footprint of the second exterior surface 222. Tilting of the access assembly 12 from the puncture position to the mounted positon may lower the pressure at which injection may begin to occur and/or increase delivery flow rate. In the example embodiment, the angle between the first exterior surface 220 and second exterior surface 222 is about 45° . Thus, transitioning the access assembly from the puncturing position to the mounted position may be accomplished by tilting of the access assembly 12 about 45°. In other embodiments, the angle between the first and second exterior surfaces 220, 222 may be adjusted. For example, the access assembly 12 may be tilted 10°, 15°, 30° to bring the access assembly from the puncture position to the mounted position and the angle between the first and second exterior surfaces 220, 222 may be selected to compel the desired amount of tilting.

Where the access member(s) 16 are microneedles, the microneedles may be oriented such that a back facing edge 23 (see also, e.g., FIG. 2) of each microneedle is the portion of the microneedle most proximal the end of the first exterior surface 220 opposite the corner 224 where the first and second exterior surfaces 220, 222 meet. As the access assembly 12 is tilted to the mounted position, the displacement path followed by the microneedle(s) may be such that the back facing edge(s) 23 may be driven through the barrier 14. The beveled surfaces leading to the back facing edge 23 may facilitate cutting of through the barrier 14 as the microneedle(s) are displaced. Thus, the backing facing edge 23 may be a cutting edge. Additionally, this may cause a face of each microneedle in which an outlet of the lumen 68 of that microneedle is disposed to be displaced away from portions of the barrier 14 contacted during the initial puncture. For example, the lumen(s) 68 of any microneedles may be displaced away from portions of the barrier 14 contacted by the sloped face(s) 21 during the initial puncture where a microneedle such as that shown in FIG. 2 is utilized. Such displacement of the microneedle(s) may aid in ensuring fluid may easily flow out of the lumen(s) 68 and into the barrier 14 (e.g. a delivery destination in a subject's skin) as delivery occurs. The above described displacement may also create a small receiving volume within the barrier 14 into which fluid may be delivered from the lumen(s) 68.

The adherence the adhesive pad 206 to the barrier may allow for delivery of agent to proceed in a hands-free manner. The adhesive pad 206 may hold the access member(s) 16 steadily in the delivery position. Where access member(s) 16 are coupled to a syringe (e.g. microneedles or standard Mantoux delivery), wobbling or other displacement of the access member(s) 16 may occur as a user attempts to hold the access members(s) 16 in position while exerting pressure to depress a plunger of the syringe. Thus, the access assembly 12 may mitigate the potential for wobbling or displacement of the access member(s) 16 within the barrier and out of the delivery position as the delivery occurs. The access assembly 12 may facilitate deliveries over relatively long periods of time without concern for displacement of the access member(s) 16 from their desired delivery positions within the barrier 14. Additionally, as example access assemblies 12 may be painless, quickly applied, and subsequently used hands-free, the access assembly 12 may facilitate shallow deliveries particularly in subjects (e.g. non-humans) which may not be receptive to being directed to remain still. This may limit needs for sedation or anesthetization during some deliveries.

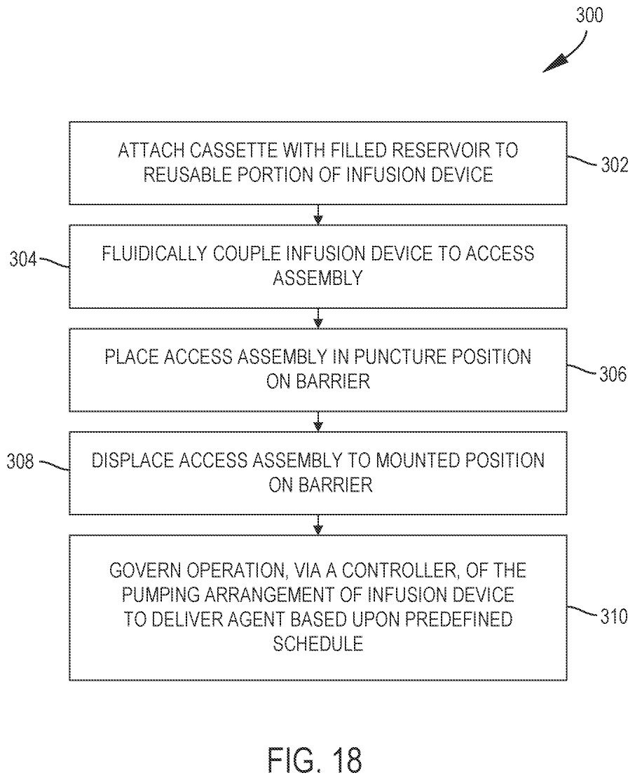

Referring now to FIG. 18, an example flowchart 300 depicting a number of actions which may be executed to deliver an agent with a system 10 including an access assembly 12 and an infusion device 18 are shown. As shown, a cassette 25 with a reservoir 22 filled with agent to be delivered may be attached to a reusable portion 27 of the infusion device 18 in block 302. The cassette 25 may be provided pre-filled or may be filled near or just before time of use (e.g. by accessing the reservoir 22 via a septum 17). In block 304, the infusion device 18 may be fluidically coupled to the access assembly 12. In certain examples, a male luer fitting associated with the infusion device 18 may be coupled to a female luer fitting associated with the access assembly 12. The access assembly 12 may be placed in a puncture position (see, e.g., FIG. 17A) in block 306. The access member(s) 16 may puncture into the barrier 14 at an angle substantially perpendicular to the barrier 14 in the puncture position and a first exterior surface 220 of an adapter 200 of the access assembly 12 may be pressed flat against the barrier 14. In block 308, the access assembly 12 may be displaced to a mounted position. An adhesive pad 206 on a second exterior surface 222 of the adapter 200 may be placed in an adhering relationship with the barrier 14 in block 308 as the access assembly 12 is brought to the mounted position. The access member(s) 16 may displace in a non-straight path within the barrier 14 as the access assembly 12 is displaced to the mounted position from the puncture position. In block 310, a controller 20 may generate commands governing operation of a pumping arrangement 24 of the infusion device. The controller 20 may operate the pumping arrangement 24 such that the infusion device 18 delivers agent to the desired shallow delivery destination based on a predefined schedule. For example, a predefined volume may be delivered over a predefined period of time or predefined volumes may be delivered over respective predefined time periods. Volumes delivered may be delivered at predefined rates.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

What is claimed is:

1. An access assembly for administration of agent to a shallow delivery destination comprising:
a body having a first exterior surface and a second exterior surface, the first exterior surface at a non-orthogonal angle to the second exterior surface, the body having a passage extending therethrough and to a corner formed between the first and second exterior surfaces, the passage defining at least one stop;
an adhesive pad coupled to the second exterior surface;
a member having a flow path extending through the member and a sharp bearing body from which a number of microneedles project coupled to an end of the member, the member disposed within the passage and in contact with the at least one stop, the at least one stop configured to inhibit displacement of the member within the passage beyond a position in which sharp bearing body is even with the first exterior surface; and
a coupler configured to couple with a cooperating coupler on a fluid flow conduit.

2. The access assembly of claim 1, wherein the angle between the first and second exterior surfaces is 10-50°.

3. The access assembly of claim 1, wherein the coupler is defined on an end of the member opposite the end to which the sharp bearing body is coupled.

4. The access assembly of claim 1, wherein the member is interference fit into the passage.

5. The access assembly of claim 1, wherein the coupler is a luer fitting.

6. The access assembly of claim 1, wherein the microneedles have a height between 200-1500 microns.

7. The access assembly of claim 1, wherein the number of microneedles is provided in a one dimensional array of microneedles.

8. The access assembly of claim 1, wherein the sharp bearing body forms an extension of the first exterior surface when the member is in contact with the stop.

9. A system for administration of agent to a shallow delivery destination comprising:
an access assembly comprising:
a body with a first exterior surface and a second exterior surface at a non-orthogonal angle to the first exterior surface, the first and second exterior surface meeting at a corner of the body;
an adhesive pad coupled to the second exterior surface;
a sharp bearing body having at least one microneedle projecting therefrom, a surface of the sharp bearing body from which the at least one microneedle projects being substantially even with the first exterior surface; and
an infusion device in fluid communication with the access assembly comprising:
a reservoir;
a pumping arrangement operable to deliver fluid form the reservoir to the access assembly;
a controller configured to govern operation of pumping assembly to deliver at least one predefined volume of agent from the reservoir to the access assembly at at least one predefined rate over at least one predefined period of time.

10. The system of claim 9, wherein the body includes a passage extending through the sharp bearing body, the sharp bearing body being coupled to a member disposed within the passage and having a flow path extending to the sharp bearing body through the member.

11. The system of claim 10, wherein the member is interference fit within the passage and the passage includes a stop which inhibits displacement of the member within the passage beyond a certain point.

12. The system of claim 9, wherein the infusion device is in fluid communication with the access device via a run of tubing.

13. The system of claim 9, wherein the at least one microneedle includes a one dimensional array of a plurality of microneedles.

14. The system of claim 9, wherein the at least one microneedle has a height dimension of 200-1500 microns. The system of claim 9, wherein the infusion device includes a volume sensing assembly configured to collect data related to the volume of agent dispensed from the infusion device.

16. The system of claim 9, wherein the access assembly includes a coupler configured to couple to a cooperating coupler in fluid communication with the infusion device.

17. The system of claim 9, wherein the infusion device includes a reusable portion and a cassette coupled to the reusable portion, the reusable portion including the controller and a first portion of the pumping arrangement, the cassette including the reservoir and a second portion of the pumping arrangement, the second portion including all components of the pumping arrangement which contact agent as the agent is dispensed.

18. A method of delivering an agent to a shallow delivery destination comprising:
placing an infusion device in fluid communication with an access assembly including at least one microneedle;
displacing the access assembly against a barrier in a puncture position in which a first exterior surface of the access assembly is pressed against the barrier and the at least one microneedle extends into the barrier;
tilting the access assembly to a mounted position in which an adhesive pad coupled to a second exterior surface of the access assembly is in a adhering relationship with the barrier, the second exterior surface meeting the first exterior surface at a corner; and
governing operation of a pumping arrangement of the infusion pump, via a controller, to deliver agent from a reservoir of the infusion device out of the at least one microneedle.

19. The method of claim 18, wherein tilting the access assembly comprises tilting the access assembly 10-50° degrees.

20. The method of claim 18, wherein the at least one microneedle extends beyond the footprint of the second exterior surface of the access assembly when the access assembly is in the mounted position.
